(19) United States
(12) Patent Application Publication
(10) Pub. No.: US 2024/0269340 A1
(43) Pub. Date: Aug. 15, 2024

(54) MICROSPHERES WITH FLOW THROUGH VOIDS AS EMBOLIC AND DRUG DELIVERY AGENTS

(71) Applicant: YISSUM RESEARCH DEVELOPMENT COMPANY OF THE HEBREW UNIVERSITY OF JERUSALEM LTD, Jerusalem (IL)

(72) Inventors: Ofra BENNY, Jerusalem (IL); Ben Zion AMOYAV, Mevasseret Zion (IL)

(21) Appl. No.: 18/569,332

(22) PCT Filed: Jun. 12, 2022
(86) PCT No.: PCT/IL2022/050622

§ 371 Date: Dec. 12, 2023

Related U.S. Application Data
(60) Provisional application No. 63/202,558, filed on Jun. 16, 2021.

(51) Int. Cl. A61L 24/04; A61K 9/50; A61L 24/00

(52) U.S. Cl. CPC A61L 24/043; A61K 9/5021; A61L 24/0015; A61L 2430/36

(57) ABSTRACT

The invention provides a novel class of microparticles suitable for inducing or causing embolism to blood microvessels.

### FIELD OF THE TECHNOLOGY

The technology disclosed herein generally concerns microspheres with flow-through voids and uses thereof.

### BACKGROUND

Hepatocellular carcinoma (HCC) is the third most common cause of cancer-related deaths worldwide, accounting for over 600,000 deaths per year. HCC comprises most primary liver cancer incidences because early detection is difficult and usually results in death within a few months of diagnosis. However, with advances in medical technology, early diagnosis is now achievable and due to the complex nature of HCC, the need for more sensitive therapeutic options is paramount. The most common treatment strategy for intermediate and advanced stage of unresectable HCC is trans-arterial chemoembolization (TACE), a procedure performed by interventional radiologists. In a typical treatment protocol, a microcatheter locally delivers anticancer agents to the tumor bed followed by administration of microspheres for arterial occlusion, resulting in ischemic tumor necrosis. Although TACE procedures can be used as a neoadjuvant therapy or as a bridging therapy to liver transplantation or resection, it suffers from several drawbacks such as inadvertent exposure of noncancerous liver tissue to cytotoxic drugs, varying particle size and the inability to release drugs in a controlled manner. This may result in liver injury, necrosis of unaffected liver tissue, and even liver failure. When the microcirculation to a tumor is blocked, oxygen levels decrease to critically low levels causing the tumor to become hypoxic. In addition, it has been revealed that the resultant period of induced acute hypoxia by TACE upregulates pro-angiogenic factors (e.g., Vascular Endothelial Growth Factor (VEGF), hypoxia-inducible factor 1-alpha (HIF-1-alpha). Hypoxic tumors are known to be chemoresistant and send outgrowth factor signals leading to angiogenesis and metastasis to other parts of the body.

In the last two decades, there have been efforts to improve the delivery of chemotherapeutic agents to tumors. Drug-eluting beads (DEB) is a relatively new drug delivery embolization system which can release the anticancer agents in a sustained manner. However, these beads characterized with high variability in diameter size, lead to wrong or inadequate blood vessel occlusion. Furthermore, DEB were associated with inducing harsh hypoxic environment that may lead to neoangiogenic reaction due to ischemia.

Benny et al [1] teach porous poly(lactic-co-glycolic) acid (PLGA) and poly(D,L-lactide) (PLA) microspheres (MPs), with varying sizes and morphologies, synthesized and optimized using both batch formulation and a flow-focusing microfluidic device. The MPs may contain an active agent and may be used to deliver such agents.

### PUBLICATIONS

[1] Benny et al., Polymers 2019, 11, 419

### SUMMARY OF THE INVENTION

In view of the state of the art, there exists a need for a biocompatible, multifunctional embolic agent for clogging, obstructing or blocking a blood vessel such as a microvessel and which can deliver various active agents, such as anticancer drugs, with high target specificity. The embolic agents of the invention, acting as solid embolic device are structured as semi free-flowing plurality of microparticles which may be provided as sponge-like microparticles, spiked microspheres or microspheres with channels on their surface, which allow for fluid flow therethrough or on their surface, and which are programmed to impose partial embolic restriction, and optionally, at a subsequent stage, full restriction, thereby inducing vessel hypoxia.

While embolic restriction may be non-selective, it is in fact dependent, inter alia, on the size and composition of the microparticles that are used and may thus be tailored to provide a sequential, step-wise and controllable restriction (i.e., starting from partial restriction and ending in full restriction), whereby several doses or amounts of various populations of microparticles may be administered, some varied in size and/or composition, while others in containing an anticancer drug. Thus, in most general terms, there is provided a provision of a novel and unique method and tools for embolic restriction of blood vessels, such as capillaries and microvessels, as means to restrict microcirculation to a tumor site.

In its most general scope, the invention concerns particles, e.g., microparticles (or sub-micron) particles, having a plurality of surface features enabling fluid flow through the microparticles or on their surface, wherein the microparticles are selected for use as embolic devices.

The invention further provides microparticles for use as embolic devices (or for use in a method of inducing embolism to a blood vessel in a subject), the microparticles being selected and structured to permit blood flow through the microparticles or on their surface and are of an average size (or a dimeter or size distribution) selected to flow into blood capillaries or microvessels in a subject's body and occlude, clog or restrict blood flow to a target tissue.

As will be further detailed hereinbelow, microparticles of the invention are based on the realization that immediate full restriction of blood flow to a target tissue, such as a cancer site, may be counterproductive as it can stimulate the tumor to secrete angiogenic factors to bypass the blockage. Partial blocking of blood flow, as rendered possible by microparticles of the invention, to a tumor does not trigger angiogenic-factor release. Together with anti-cancer treatment protocols they may decrease a tumor load. Furthermore, allowing partial blood flow to a tumor site allows treating the tumor with drugs carried by the blood, either drugs provided systemically or drugs released from the particles themselves.

In most general terms, and as further elaborated herein, the particles used as embolic devices may be any one or more of the following particles populations:
(1) Microparticles or sub-micron particles having flow through features, wherein the microparticles are free of or not associated with any active or diagnostic agent;
(2) Microparticles or sub-micron particles having flow through features, wherein the microparticles are loaded with or associated to one or more active or diagnostic agents;
(3) Microparticles or sub-micron particles having surface features, such as spikes or grooves, wherein the microparticles are free of or not associated with any active or diagnostic agent;
(4) Microparticles or sub-micron particles having surface features, such as spikes or grooves, wherein the microparticles are loaded with or associated to one or more active or diagnostic agent;
(5) Core/shell microparticles having each a core and a shell, wherein the core is of a solid material which may or may not be biodegradable and wherein the shell having flow through features or surface features that permit blood flow through the shell or on the microparticles surface, wherein the microparticles are free of or not associated with any active or diagnostic agent;
(6) Core/shell microparticles, having each a core and a shell, wherein the core is of a solid material which may or may not be biodegradable and wherein the shell having flow through features or surface features that permit blood flow through the shell or on the surface of the microparticles, wherein the microparticles are loaded with or associated to one or more active or diagnostic agent;
(7) Microparticles surface decorated with one or more nanoparticles, wherein the microparticles and/or the nanoparticles are free of or are not associated to active or diagnostic agents;
(8) Microparticles surface decorated with one or more nanoparticles, wherein the microparticles and/or the nanoparticles are loaded with or associated to one or more active or diagnostic agents;
(9) Particles as defined hereinabove loaded with associated to one or more active or diagnostic agents;
(10) Collapsible or erodible forms of any of the above mentioned microparticles; and/or
(11) Mixtures of the above microparticle populations.

Microparticles used in formulations, devices and methods herein are not liposomes, micellar structures, nor particulate forms having any vesicle structures. Also excluded are any particle formed of or containing phospholipids and metallic particles.

In some embodiments, each of the microparticles is provided with flow through features, such as channels, that allow for partial blood flow through the particles body. As will be further explained herein, the flow through features are not surface pores.

In some embodiments, each of the microparticles is provided with surface decorated features, such as spikes or channels or grooves, that allow for partial blood flow on the particles' surface.

In some embodiments, each of the microparticles is provided with flow through features, such as channels, and surface features, such as spikes or channels or grooves, that allow for partial blood flow through the particles body, as well as on their surface.

In some embodiments, each of the microparticles is provided in a collapsible or erodible form and/or composition that allow for reduction in the microparticles' size to a size permitting more severe restriction (partial or complete) of blood microvessels as compared to the microparticles' original or pre-collapsed form.

In some embodiments, the microparticles have a first predetermined average particle size and are configured to erode (or collapse) to a second average particle size being smaller than the first predetermined average size. In such configurations, when delivered to a lumen of a blood vessel, the microparticles are of an average particle size (first predetermined size) and structure porosity that partially interrupts blood flow through the vessel at a first position of the vessel having a first width. Yet, with time, erosion causes reduction in particle size (to a second particle size), which results in the smaller particle being carried by the blood stream to a second more narrow position in the blood vessel and obstructing it altogether, thus causing embolism.

Also provided are collapsible microparticles of a first size (diameter) ranging between 10 and 500 μm and having a plurality of flow-through features and/or surface features, e.g., voids or channels and/or spikes or grooves, enabling blood flow through and/or around the features, wherein the microparticles are structured or configured to collapse and/or erode to microparticles of a second size being between 4 and 50 μm.

As may be understood, a microparticle having a first size or diameter within the range of 10 and 500 μm may erode to a microparticle having a second size or diameter within the range of 4 and 50 μm. For example, a microparticle of a first size or a diameter of 100 μm may erode or reduce in size to a microparticle having a second size or a diameter that is within the initial range (i.e., of the first size), but also within the second range of 4 and 50 μm, say 40 μm. Similarly, a microparticle having a first size or diameter of 45 μm (being within the range of the first size or diameter) may erode to a size or diameter of 12 μm (being within the range of the second size or diameter).

In some embodiments, the microparticles of the invention are collapsible particles, namely they are structured to structurally break down to reduce in size and/or volume. The particles are alternatively or additionally erodible, meaning they wear down or lose material or degrade or biodegrade due to any mass-movement processes which may involve mechanical or chemical/biological processes, triggered or caused by flow of the microparticles and/or contacting of the microparticles with a variety of chemical/biological components present in the blood system, e.g., enzymes. The mechanical process may involve friction, mechanical mass loss due to contact with vessel walls or tissues, or due to any contact-degradation process. Chemical degradation processes may involve biodegradation or hydrolytic degradation or any other such degradation of a microparticle material, e.g., polymeric material, that has a predetermined solubility or (bio)degradation rate in vivo. Thus, the term “collapsibility and/or erosion” or any lingual variation of the expression may encompass any mass-movement or mass-reduction or physical structural deformation processes which causes particle size reduction.

The microparticles of the invention may or may not be porous. If porosity exists, it is always secondary to the presence of flow through features and/or surface features that must be present in order to allow partially restricted blood flow through the microparticles or on their surface. The microparticles may however be sponge-like, spherical particles, and may adopt any shape and size within the indicated size or diameter range. In some cases, the microparticles are of amorphous shapes (namely of irregular random shapes) or may adopt a single or substantially single non-spherical shape, such as an elliptical shape or another irregular shape.

Irrespective of the material composition, the process of mass-degradation or mass-change, and particles shape, the microparticles may have a first or an initial average size or dimeter ranging between 10 and 500 μm. In some embodiments, the average particles size or diameter may be between 10 and 450 μm, 10 and 400 μm, 10 and 350 μm, 10 and 300 μm, 10 and 250 μm, 10 and 200 μm, 10 and 150 μm, 10 and 100 μm, 10 and 50 μm, 20 and 500 μm, 30 and 500 μm, 40 and 500 μm, 50 and 500 μm, 60 and 500 μm, 70 and 500 μm, 80 and 500 μm, 90 and 500 μm, 100 and 500 μm, 150 and 500 μm, 200 and 500 μm, 250 and 500 μm, 300 and 500 μm or between 350 and 500 μm.

In some embodiments, the microparticles provided for use according to aspects and embodiments of the invention are of an average first size or diameter ranging from 50 and 150 μm, 60 and 150 μm, 70 and 150 μm, 80 and 150 μm, 80 and 200 μm or between 80 and 250 μm.

In some embodiments, the particles are submicron particles having a dimeter or a size that ranges between 100 and 500 nm. In some embodiments, the average particles size or diameter may be between 100 and 450 nm, 100 and 400 nm, 100 and 350 nm, 100 and 300 nm, 100 and 250 nm, 100 and 2000 m, 100 and 150 nm, 200 and 500 nm, 300 and 500 nm, 400 and 500 nm, 250 and 500 nm, 300 and 500 nm or between 350 and 500 nm.

In some embodiments, the particles used for purposes herein are not nanoparticles.

The diameter or size of microparticles of the invention may be determined by manufacturing particles with a size distribution limitation that meets the intended uses. Manufacturing processes as well as tools of measuring particles sizes or size distributions are well known in the art. Where the microparticles are substantially spherical, the size or diameter may be measured from the center or through the center of the microparticles. In cases where the microparticles are core/shell, their size or diameter may similarly be measured from or through the center of the microparticle to the outermost particle surface or circumference. Where the microparticles are elliptical or non-spheroid, the size provided herein refers to their longest axis. In cases where the particles are spiked or have surface hooks or other decorations, the size or diameter will be the distance measured from one hook or spike end, through the center of the microparticle to the other hook or spike end. Thus, where the microparticles are spiked, erosion of their spikes may yield particles of smaller size that can meet the intended purposes as disclosed herein.

In embolic devices of the invention, material lose or erosion to yield the smaller sized microparticles is designed to provide microparticles with an average size that does not restrict or constrict blood flow in large blood vessels but does restrict smaller blood vessels, particularly capillaries or microvessels found in tumors. The inner diameter of such capillaries or microvessels may range between 4 and 10 μm. Thus, microparticles of predetermined sizes or such that are configured to undergo erosion or material loss or degradation or collapse to predetermined sizes should provide embolic microparticles of an average size suitable for contracting blood capillaries or blood microvessels, resulting in ischemic tissue, e.g., tumor necrosis.

Thus, in some embodiments, the microparticles may be of a size that is small enough to cause clogging or blocking of a capillary or a microvessel, a size being, in some embodiments, between 4 and 50 μm. In some embodiments, the microparticles have an average size of between 4 and 10 μm, 4 and 20 μm, 4 and 30 μm, 4 and 40 μm, 10 and 20 μm, 20 and 30 μm, 30 and 40 μm, 40 and 50 μm, 10 and 40 μm, 10 and 30 μm, 20 and 50 μm, 20 and 40 μm, 30 and 50 μm, or 4, 5, 6, 7, 8, 9, or 10 μm. As microparticles of the invention may be administered to meet one or more clinical purposes, their size, or in some embodiments, their initial size and final size (e.g., before and after collapse and/or erosion) may be predetermined by selecting, inter alia, the microparticles material composition, their structure (core/shell, spiked, etc) and others. A medical practitioner will know to administer microparticles of a certain composition and size, knowing well that their size may reduce to a size that is capable of clogging or closing a vessel, as disclosed herein.

One versed in the art would realize that microparticles of a size of e.g., 50 μm, having flow through features or surface decorations, as disclosed herein, may be suitable as embolic devices even without material collapse, degradation or erosion.

The microparticles are typically polymeric particles that are composed of, comprised of or consist at least one polymeric material. However, the microparticles may be made of other non-polymeric materials. In some embodiments, the microparticles are composed or comprise or consist a biodegradable or biodegradable polymeric material. Non-limiting examples of polymeric materials from which microparticles of the invention may be made of include poly (lactic-co-glycolic) acid (PLGA), poly (D,L-lactide) (PLA), polycaprolactone (PCL), poly(methyl methacrylate) (PMMA), poly(vinylacetate), polystyrene diblock copolymers, polymerized high internal phase emulsion (polyHIPE), polyvinyl alcohol (PVA), poly(N-isopropylacrylamide) (PNIPAAm) and natural polymers such as collagen, cellulose, alginate and gelatin.

In some embodiments, population of microparticles are used, wherein each population comprises or consists microparticles of different polymeric materials.

In some embodiments, the microparticles comprise or consist PLGA, PLA or PCL.

For some applications, microparticles of the invention may be provided as core/shell structures having each a solid (non-porous) core and a shell providing a plurality of flow-through voids (wherein the shell is a sponge-like structure) and/or surface features (spikes, channels/grooves as described above) enabling flow of blood through the shell region and/or its surface. In such cases, the core may be made of materials that are not porous and/or not biodegradable or not biodegradable. Such core materials may be polymeric or non-polymeric in composition. Non-limiting examples include polystyrene, silica, glass, polyethylene, polycarbonate, polyurethane, high molecular weight of the above-mentioned polymers or any other inert material.

In some embodiments, erosion or degradation of the shell material may produce smaller microparticles (having a second size) which can move further into the blood vessel and cause constriction of narrower blood vessels on the way. In such embodiments, the polymer of the shell may be selected to undergo erosion or mass loss at a rate that is greater than the degradation rate of the core polymer. Alternatively, the polymer of the shell may be selected to undergo degradation or mass loss, while the core polymer is not biodegradable.

Generally speaking, the shell, under physiological conditions, degrades before the core. Degradation rate can be tailored by selecting the type of polymer; the polymer chains size and polymer molecular weight. Thus, the core and the shell regions may be composed of the same polymeric material, but each region may be characterized by a different rate of degradation. In some embodiments, the core and shell are of different polymeric materials. The core can be made of a non-degradable polymer, e.g. polystyrene, or silica or other inert polymers and the outer layer may be of a biodegradable or an erodible material. In other embodiments, the core and shell are of the same polymeric material.

In some embodiments, the core and shell are of the same or different polymeric materials, but the shell is structured or tailored to provide porosity, while the core may not be porous.

Thus, microparticles of the invention may be provided in preselected sizes and compositions, and thus may be used as embolic devices or microparticles having two defined states: a first state—in a form allowing blood flow through the blood vessels, the form being administered to the blood stream, and a second state—a blood flow interrupting form, wherein the microparticles are reduced in size. The microparticles transform from the first to the second form in the body. When initially presented to the body or blood stream, i.e., by parenteral administration, the microparticles, having flow-through voids or microchannels, enable partial or uninterrupted flow of fluids, i.e., blood components, solutes, serum and the like, over their surface and through the plurality of voids. As time passes, the polymeric shell or polymeric surface of the microparticles erodes or degrades losing their surface voids or features. These smaller in size microparticles, typically 5 to 50 microns in size, are capable to flow or move with the blood stream into narrow blood vessels which they now clog, thereby causing tumor necrosis.

A general concept of the invention is depicted in FIG. 1.

In some embodiments, the invention also concerns microparticles of a size (diameter) ranging between 10 and 500 μm and having a plurality of flow-through voids enabling blood flow through the voids or on the microparticles surface, wherein the microparticles are composed of a biodegradable or biodegradable material.

In some embodiments, the microparticles are not core/shell particles, but composed of a single polymeric material. In some embodiments, the microparticles are not core/shell particles and composed of a single material wherein the outer surface of the microparticles having the plurality of flow-through voids.

In some embodiments, the microparticles are not core/shell particles, but composed of a single or a blend of polymeric material(s), wherein the different regions of the microparticles are inseparable from others; namely no core and/or shell features.

In some embodiments, the microparticles are core/shell particles, wherein the core and the shell regions are made of the same or different materials. In some embodiments, the core and shell are made of different materials, each selected as indicated herein and wherein the shell comprises the flow-through voids. In some embodiments, the core is not porous, or is not biodegradable or is not biodegradable.

In some embodiments, the microparticles are Janus microspheres having at least two distinct physical or chemical properties. The Janus particles may be used for drug combinations, for example hypoxia-activated drug may be combined with chemotherapy or an angiogensis inhibitor may be combined with a cytotoxic drug, or an imaging agent may be combined with a drug. The use of Janus particles also allows controlling different or distinct release kinetics in cases where two polymers are used, e.g., a polymer such as PLGA with different molecular weights.

The invention also concerns microparticles of a size (diameter) ranging between 10 and 500 μm and having a plurality of flow-through voids configured to allow for a liquid flow, e.g., blood flow, through the voids or on the microparticles surface, wherein the microparticles are composed of a biodegradable or biodegradable material permitting size diminution in the body to particles of a size ranging between 5 and 50 microns. In some embodiments, size diminution occurs over a period of between a few hours and couple of days.

Microparticles of the invention may be provided substantially spherical with a degree of surface porosity and flow-through voids, or additionally or alternatively, be provided with a plurality of surface features such as anchoring features, e.g., spikes. These anchoring surface features or spikes or hooks permit anchoring to blood vessel walls. The spikes extend the circumference of the microparticles to a length typically in the range of at least about 10% of the diameter of the particle. The shape of the spikes may be needle like, conical or irregular. The spikes may be of the same polymer of the (solid) core or may be composed of a second polymer. In some embodiments, the spike elements may be biodegradable or biodegradable.

Typically, the spikes are arranged on the circumference of the particles at a density that is variable and at times uncontrolled. The distribution on the particles surface may have limited effect on the functionality of the microparticles as occluders.

Irrespective of the shape and composition of the microparticles, the microparticles are structured or provided with a plurality of surface features and/or flow-through void features which permit continuous flow of blood through the particles matrix or bulk or on their surface. Such a blood flow may be maintained uninterrupted, albeit restricted blood flow in blood vessels which full clogging or obstruction is not desired or is to be avoided, e.g., large blood vessels such as arteries and veins. The surface features or void features are thus features which enable continuous flow of blood or blood components through the microparticles or on their surface. The features may be channel-like voids or structures that transverse at least a part of the volume or body of the microparticle. These may be in the form of interconnecting pores that allow for a continuous liquid flow through (tortuous) the channel that extends a distance between the pores. In microparticles having surface spikes, uninterrupted flow may be maintained by providing the spikes at such a surface density which allows fluid or blood to flow uninterruptedly between the spikes. Microparticles that become anchored to a blood vessel inner walls do not therefore interrupt blood flow. In such configurations, the distances between the spikes define flow-through voids, which for some cases renders unnecessary presence of the channel-like voids or structures detailed above.

Thus, the term “flow-through void” refers to any such feature that is present in the microparticles or on their surface that maintains blood flow (albeit partially restricted) through the particles or on a region between its surface and the inner wall of the blood vessel. Surface pores that are not interconnecting, namely pores which are simply surface dentations or holes do not constitute flow-through voids.

Particles of the invention may be provided bear or free of any drug or diagnostic agent, or may be provided in a loaded form, wherein at least one drug or a diagnostic agent is provided, contained, encapsulated in or associated to the microparticle bulk material or surface. As microparticles of the invention may be structured of two or more features, e.g., a shell, spikes, etc, and/or two or more materials, e.g., a core material and a shell material, and/or cavities or pockets in which a drug or a diagnostic agent may be contained, the selection of drugs or diagnostic agents may be amongst labile and stable active materials, hydrophilic and lipophilic active materials, drugs or agents of low or high molecular weights, drugs or agents of varying Log P values, etc.

As used herein, the microparticles may be “loaded with” or “associated to” one or more drug or a diagnostic agent. The drug or diagnostic agent may be contained within the microparticle, namely in a core thereof or in a material making up the microparticle body or may be associated with its surface or bulk material via chemical or physical anchoring. Irrespective of the means or form by which the drug or diagnostic agent is/are contained or carried by the microparticle, the terms do not mean to suggest loading or association of a particular amount of an active or a diagnostic agent. Any amount may be used, said amount may be determined by a variety of factors including, inter alia, the size of the microparticles, their material composition, the drug or agent attributes (hydrophilic and lipophilic drugs, drugs of low or high molecular weights, drugs of varying Log P values, etc), a desired dosage, type of release (slow, immediate etc) and others.

In some embodiments, the microparticles, structured of a single material, such as a polymeric material, comprise one or a mixture of drugs contained within the particle material.

In some embodiments, the microparticles are core/shell structures, containing one or more drugs. In some embodiments, one drug may be contained in the core, while another different drug may be contained in the shell.

In some embodiments, the microparticles are core/shell structures, containing same drug in the core and in the shell regions, allowing controlled or timed release based on the relative degradation profiles of the shell and the core.

In some embodiments, the microparticles are spiked or are surface decorated with tissue attaching features or hooks, wherein said features or hooks may be comprised of a fast-degrading material. In such embodiments, the microparticle core may comprise one type of a drug while the features or hooks may contain another.

In some embodiments, the microparticles surface decorated or surface associated with one or a plurality of nanoparticles. The microparticles and the nanoparticles associated with the microparticles' surface may be of the same or different materials. Their sizes and shapes may vary, as defined herein. In some embodiments, the decorating nanoparticles are composed of materials different from the microparticle, thus permitting, for example, drug loading of different drug entities.

In some embodiments, the microparticles are surface decorated or surface associated with one or a plurality of nanoparticles configured to dissociate into a main microparticle and a plurality of nanoparticles, such that the microparticle an each of the nanoparticles are designed to provide a different benefit in situ. For example, the nanoparticles may carry a drug or a diagnostic agent into smaller blood vessels feeding a tumor, while the microparticle may be used to induce embolism.

Typically, the decoration by the nanoparticles allows administration of drugs or diagnostic agents which are chemically non-compatible with the polymers of the large microparticles. Typically, the large microparticle may incorporate hydrophobic drugs or diagnostic agents, while the smaller nanoparticle may incorporate hydrophilic drugs or diagnostic agents.

The “drug” may be any active agent which delivery into a blood vessel to be occluded may promote, induce, cause or provide a medical benefit. In some embodiments, the drug is optionally selected amongst cytotoxic agents or cytostatic agents, wherein cytotoxic agents prevent cancer cells from multiplying by: (1) interfering with the cell's ability to replicate DNA; and (2) inducing cell death and/or apoptosis in the cancer cells, while the cytostatic agents act via modulating, interfering or inhibiting the processes of cellular signal transduction which regulate cell proliferation. Notwithstanding the mechanism of action, the drug may be any anticancer drug, cytotoxic agent, a drug that selectively acts in hypoxic tumors, an anti-angiogenic agent, an anti VEGF agent, an antimetabolite, a topoisomerase inhibitor, a protein tyrosine kinase inhibitor, a proteasome inhibitor and others.

Non-limiting examples of cytotoxic agents suitable for use in microparticles of the invention include
antimetabolites, such as capecitibine, gemcitabine, 5-fluorouracil, leucovorin, fludarabine, cytarabine, mercaptopurine, thioguanine, pentostatin, and methotrexate;
topoisomerase inhibitors, such as etoposide, teniposide, camptothecin, topotecan, irinotecan, doxorubicin, and daunorubicin;
vinca alkaloids, such as vincristine and vinblastin;
taxanes, such as paclitaxel and docetaxel;
platinum agents, such as cisplatin, carboplatin, and oxaliplatin;
antibiotics, such as actinomycin D, bleomycin, mitomycin C, adriamycin, daunorubicin, idarubicin, doxorubicin and pegylated liposomal doxorubicin;
alkylating agents such as melphalan, chlorambucil, busulfan, thiotepa, ifosfamide, carmustine, lomustine, semustine, streptozocin, decarbazine, and cyclophosphamide;
protein tyrosine kinase inhibitors, such as imatinib mesylate and gefitinib;
proteasome inhibitors, such as bortezomib;
antibodies, such as trastuzumab, rituximab, cetuximab, and bevacizumab;
others as known in the art.

In some embodiments, the therapeutic drug is selected from altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, carboplatin, cisplatin, eptaplatin, miriplatine hydrate, oxaliplatin, lobaplatin, nedaplatin, picoplatin, satraplatin, lobaplatin, nedaplatin, picoplatin, satraplatin, amrubicin, bisantrene, decitabine, mitoxantrone, procarbazine, trabectedin, clofarabine, amsacrine, brostallicin, pixantrone, laromustinel, etoposide, irinotecan, razoxane, sobuzoxane, teniposide, topotecan, amonafide, belotecan, elliptinium acetate, voreloxin, cabazitaxel, docetaxel, eribulin, ixabepilone, paclitaxel, vinblastine, vincristine, vinorelbine, vindesine, vinflunine; fosbretabulin, tesetaxel, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur, doxifluridine, elacytarabine, raltitrexed, sapacitabine, bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin, aclarubicin, peplomycin, pirarubicin, abarelix, abiraterone, bicalutamide, buserelin, calusterone, chlorotrianisene, degarelix, dexamethasone, estradiol, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol, acolbifene, danazol, deslorelin, epitiostanol, orteronel, aminoglutethimide, anastrozole, exemestane, fadrozole, letrozole, testolactone, formestane, crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib, talaporfin, temoporfin, alemtuzumab, besilesomab, brentuximab vedotin, cetuximab, denosumab, ipilimumab, ofatumumab, panitumumab, rituximab, tositumomab, trastuzumab, bevacizumab, pertuzumab, catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab, nivolumab, denileukin diftitox, ibritumomab tiuxetan, iobenguane, prednimustine, trastuzumab emtansine, estramustine, gemtuzumab, ozogamicin, aflibercept, edotreotide, inotuzumab ozogamicin, naptumomab estafenatox, oportuzumab monatox, and others.

In some embodiments, the drug is an anticancer drug, such a doxorubicin.

In some embodiments, the drug is a hypoxic cytotoxin selected to act on tumors in hypoxic state (oxygen deprived state). Such drugs may be selected from tirapazamine (TPZ), banoxantrone (AQ4N), porfitomycin, apaziquone (EO9), 1,2-bis(methylsulfonyl)-1-(2-chloroethyl)-2-[[1-(4-nitrophenyl)ethoxy]carbonyl]hydrazine (KS 119), dinitro benzamide mustard derivative (such as PR 104) and 4-[3-(2-nitro-1-imidazolyl)-propylamino]-7-chloroquinoline hydrochloride (NLCQ-1, NSC 709257).

In some embodiments, the microparticles are provided with two or more drugs, one being an anticancer drug and another a hypoxic cytotoxin. In some embodiments, the anticancer drug is doxorubicin and the hypoxic cytotoxin is TPZ.

In some embodiments, the microparticles are loaded or associated with a diagnostic agent. The diagnostic agent may be an imaging material of any type known in the art. It may be a contrasting agent, a radiopharmaceutical and others. Nonlimiting examples of such agents include X-ray contrasting agents, e.g., magnetite, iron-containing materials and Lipiodol; magnetic resonance imaging agents, such as gadoterate, gadobutrol, gadoteridol, gadopentetate, gadobenate, gadopentetic acid dimeglumine, gadoxentate, gadoversetamide, gadodiamide, albumin-binding gadolinium complexes, gadofosveset, gadocoletic acid, polymeric gadolinium complexes, gadomelitol, gadomer, gadoxetic acid and others; ultrasound contrast agents such as microbubbles, perflutren lipid microspheres, octafluoropropane gas core with an albumin shell microbubbles, sulfur hexafluoride microbubbles, air and a lipid/galactose shell microbubbles, perflexane lipid microspheres and others.

In some embodiments, the diagnostic agent is lipiodol.

In some embodiments, the drug is at least one biologically active agent that is suitable as a drug, a diagnostic agent or an imaging agent.

The biologically active agent, alone or in combination with another active or non-active agent, may be coated on the surface of the microparticle, may be present in a core of a microparticle, or may be impregnated in a material constituting the nanoparticle. For hydrophobic compounds, the drug may be incorporated inside the polymer skeleton by adding the substance during the preparation method to the “oil” phase, which is also the polymer solvent. Alternatively or additionally, the microparticles may be surface associated with a drug or generally with an active entity via a surface linker moiety, by absorption to the surface or by intercalation of physical attachment to the surface.

Where the biological agent is intended to be used as a diagnostic or as an imaging agent, it may be present in the particle in a releasable or a non-releasable form. Where the biologically active agent is a drug, it is typically present in the particle in a manner enabling its release after administration, preferably in a controlled or sustained release manner. The release may be induced by a homogenous degradation triggered by the aqueous environment, or by surface erosion. Once mechanical properties of the polymer are weakened, or the polymer erodes, the drug may diffuse out or may be released.

Non-limiting examples of agents that may be used include anticancer agents and active agents as detailed herein; diagnostic agents such as X-ray contrasting agents, e.g., magnetite, iron-containing materials and Lipiodol; microbubbles and others, as disclosed herein.

The invention further provides microparticles according to the invention for use in clogging a blood vessel, e.g., typically a blood capillary or a microvessel.

The invention further provides a pharmaceutical or diagnostic formulation comprising an effective amount of particles according to the invention.

The invention further provides a formulation or a suspension or a dispersion suitable for administration to a human or animal subject, the suspension or dispersion comprising a plurality of particles according to the invention suspended or dispersed in a saline solution.

In some embodiments, the formulation or suspension or dispersion is provided as an intravenous fluid, e.g., in an infusion bag.

The invention thus provides an intravenous fluid, e.g., an infusion bag, comprising a formulation or a suspension or a dispersion as defined herein.

Formulations of the invention, as are microparticles of the invention, are adapted for parenteral administration, namely adapted for non-oral administration. Typically, the formulations are adapted for intramuscular (IM), subcutaneous (SC) and intravenous (IV) administrations. In some embodiments, parenteral administration is IV, e.g., arterial/vein guided injection by syringe or catheter.

The invention further provides a pharmaceutical formulation for IV administration, the formulation comprising an effective amount of particles according to the invention, for use in methods of occluding a blood vessel or for delivering an active agent to said vessel over a period of time, without undergoing degradation or infiltration through a vessel wall.

In some embodiments, the microparticles are provided as a population of microparticles which may be homogenous or may be heterogeneous. In some embodiments, the population is a mixed population composed of microparticles with flow-through voids, as disclosed herein, and microparticles free of such voids. In some embodiments, microparticles which are free of flow-through voids may be surface decorated with spikes, as disclosed herein.

The microparticles population may comprise a single population of microparticles or two or more populations of microparticles. In some embodiments, the population may comprise at least two microparticles populations, wherein each population is different from another in at least one of microparticle size, microparticle structure, density of surface features, structure of the flow-through voids, microparticles composition and presence or absence of an active agent.

The microparticles may be administered in a form suitable for pharmaceutical administration and use. A pharmaceutical composition comprising the microparticles may further comprise a pharmaceutically acceptable carrier, a vehicle or an adjuvant and may be further adapted based on the intended use, e.g., for parenteral administration.

Bare microparticles of the invention, as well as drug-loaded microparticles, may be utilized in a variety of therapeutic methods. For example, the microparticles may be administered for the treatment of cancer as part of transarterial chemoembolization (TACE). In such a utility, the drug may be an anti-cancer drug used in chemotherapy, such as those mentioned hereinabove, e.g., doxorubicin, cisplatin and mitomycin, as well as other anti-cancer drugs such as immunomodulatory drugs, anti-angiogenic agents, such as sorafenib and avastin and others.

The present invention thus also concerns a method of treatment utilizing administration of microparticles of the invention.

The invention provides in an aspect thereof a method of killing cancer cells or a tumor in a subject, the method comprising administering to said subject by parenteral administration a formulation comprising microparticles of the invention.

The invention further provides a method for inducing embolism to a blood vessel, e.g., a microvessel, in a subject, the method comprising administering to said subject by parenteral administration a formulation comprising microparticles of the invention.

The present invention further provides a method of transarterial chemoembolization (TACE), the method comprising administering to a blood vessel of a subject a therapeutically effective amount of microparticles as disclosed herein.

The invention further provides a method of selective eradication or killing or causing death to tumor cells in a subject, the method comprises causing hypoxia to said tumor cells or tissue containing same and administering to said subject (1) a population of microparticles according to the invention, the population of microparticles comprising microparticles loaded with at least one hypoxia-activated agent; or (2) a population of microparticles according to the invention, and subsequently thereto administering at least one hypoxia-activated agent; wherein the hypoxia activated agent becomes activated for eradicating the tumor cells at the region of hypoxia within a microvessel to the tumor.

In some embodiments, hypoxia is caused by administering embolic devices, i.e., microparticles, according to the invention.

The invention further provides a method of selective eradication or killing or causing death to tumor cells in a subject, the method comprises administering to said subject a population of microparticles according to the invention, the population of microparticles comprising microparticles loaded with at least one hypoxia-activated agent; or administering to said subject a population of microparticles according to the invention, and subsequently thereto administering at least one hypoxia-activated agent; wherein the hypoxia activated agent becomes activated for eradicating the tumor cells at the region of hypoxia within a microvessel to the tumor.

In some embodiments, the subject is administered a population of microparticles according to the invention, the population of microparticles comprising microparticles loaded with at least one hypoxia-activated agent.

In some embodiments, the subject is administered with a population of microparticles according to the invention, and after a period of time, is administered with at least one hypoxia-activated agent. The period of time spanning between the two administration protocols may be several hours to several days.

In some embodiments, the at least one hypoxia-activated agent contained in microparticles of the invention or administered separately is at least one hypoxic cytotoxin, such as tirapazamine (TPZ), banoxantrone (AQ4N), porfitomycin, apaziquone (EO9), 1,2-bis(methylsulfonyl)-1-(2-chloroethyl)-2-[[1-(4-nitrophenyl)ethoxy]carbonyl]hydrazine (KS 119), dinitrobenzamide mustard derivative (such as PR 104) and 4-[3-(2-nitro-1-imidazolyl)-propylamino]-7-chloroquinoline hydrochloride (NLCQ-1, NSC 709257).

In some embodiments, the at least one hypoxia-activated agent is administered in combination with at least one anticancer drug, as defined herein.

The microparticles used in methods of the invention may be selected amongst:
(1) Microparticles or sub-micron particles having flow through features, wherein the microparticles are free of or not associated with any active or diagnostic agent;
(2) Microparticles or sub-micron particles having flow through features, wherein the microparticles are loaded with or associated to one or more active or diagnostic agents;
(3) Microparticles or sub-micron particles having surface features, such as spikes or grooves, wherein the microparticles are free of or not associated with any active or diagnostic agent;
(4) Microparticles or sub-micron particles having surface features, such as spikes or grooves, wherein the microparticles are loaded with or associated to one or more active or diagnostic agent;
(5) Core/shell microparticles having each a core and a shell, wherein the core is of a solid material which may or may not be biodegradable and wherein the shell having flow through features or surface features that permit blood flow through the shell or on the microparticles surface, wherein the microparticles are free of or not associated with any active or diagnostic agent;
(6) Core/shell microparticles, having each a core and a shell, wherein the core is of a solid material which may or may not be biodegradable and wherein the shell having flow through features or surface features that permit blood flow through the shell or on the surface of the microparticles, wherein the microparticles are loaded with or associated to one or more active or diagnostic agent;
(7) Microparticles surface decorated with one or more nanoparticles, wherein the microparticles and/or the nanoparticles are free of or are not associated to active or diagnostic agents;
(8) Microparticles surface decorated with one or more nanoparticles, wherein the microparticles and/or the nanoparticles are loaded with or associated to one or more active or diagnostic agents;
(9) Particles as defined hereinabove loaded with associated to one or more active or diagnostic agents;
(10) Collapsible or erodible forms of any of the above mentioned microparticles; and/or
(11) Mixtures of the above microparticle populations.

In some embodiments, the microparticles or sub-micron particles have flow through features, wherein the particles are free of any active or diagnostic agent.

In some embodiments, the microparticles or sub-micron particles have flow through features, wherein the particles are loaded with one or more active or diagnostic agents.

In some embodiments, the microparticles or sub-micron particles have surface features such as spikes, wherein the particles are free of any active or diagnostic agent.

In some embodiments, the microparticles or sub-micron particles have surface features such as spikes wherein the particles are loaded with one or more active or diagnostic agent.

In some embodiments, the microparticles are core/shell microparticles, wherein the core being of a solid material which may or may not be biodegradable and wherein the shell having flow through features or surface features that permit blood flow through or on the surface of the microparticles, wherein the microparticles are free of any active or diagnostic agent.

In some embodiments, the microparticles are core/shell microparticles, wherein the core being of a solid material which may or may not be biodegradable and wherein the shell having flow through features or surface features that permit blood flow through or on the surface of the microparticles, wherein the microparticles are loaded with one or more active or diagnostic agent.

In some embodiments, the microparticles are surface decorated with one or more nanoparticles, wherein the microparticles and/or the nanoparticles are free of active or diagnostic agents.

In some embodiments, the microparticles surface are decorated with one or more nanoparticles, wherein the microparticles and/or the nanoparticles are loaded with one or more active or diagnostic agents.

In some embodiments, the microparticles as defined herein are associated with one or more active or diagnostic agents.

In some embodiments, the microparticles are collapsible or erodible forms of any one of the microparticles disclosed herein.

In some embodiments, the microparticles are provided as mixed particle populations.

In some cases, bare microparticles may be used for achieving an effective TACE, wherein after administration of the microparticles, they undergo structure collapse, biodegradation or bioerosion and lose their outer layer(s) (including the surface features: voids or spikes), resulting in particles of smaller sizes or diameters. Advancing through blood vessels the smaller particles completely block blood flow. The reduction in particle size causes an initial partial obstruction (while not inducing hypoxia in the tumor tissue) followed by complete obstruction and cell death.

The “effective amount” of microparticles used in a treatment protocol for causing embolism or for delivering an agent to a target cancerous tissue may depend on a variety of factors, such as presence or absence of active ingredients, the size and shape of the microparticles, the mode of action, the particles degradation profile etc. Generally, the effective amount may be any such amount that is sufficient to partially or fully clog or embolize blood flow to a tumor via microvessels or capillaries occlusion, or that is sufficient to induce hypoxia to said tissue. Tumor toxicity and toxicity to normal tissues and cells, as well as therapeutic efficacy can be determined by standard pharmaceutical procedures in vitro, in cell cultures or experimental animals.

The dosage or effective amount may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1).

Cancers that can be treated by methods and products of the invention include cancer of the liver, prostate, uterus, kidneys; hepatoma or hepatocellular carcinoma (primary liver cancer); holangiocarcinoma (primary cancer of the bile ducts in the liver); metastasis (spread) to the liver from colon cancer; breast cancer; carcinoid tumors and other neuroendocrine tumors; islet cell tumors of the pancreas; ocular melanoma; sarcomas; other vascular primary tumors in the body.

The invention also concerns a method of sustained delivery of a drug or a diagnostic agent to a subject, the method comprises administering to the blood vessels of the subject in need of treatment by the drug, a therapeutically effective amount of microparticles comprising said drug or agent.

The invention further provides a kit comprising a plurality of microparticles of the inventions and instructions of use.

In some embodiments, in a kit of the invention, the microparticles are provided as a powder or in an appropriate emulsion, dispersion or suspension.

In some embodiments, the microparticles are provided in a form suitable for mixing or formulating into a pharmaceutical composition.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 provides an illustration of the general concept of the technology disclosed in the present application.

FIG. 2 depicts porous microspheres fabricated with microfluidic features according to some embodiments of the invention.

FIGS. 3A-D depicts an embolism flow chip device according to some embodiments of the invention. (FIG. 3A) shows a 3D printed mold of the embolization microfluidic chip, wherein (A) is a porous particles entrance and (B) is a Hep G2 liver HCC cell line entrance. (FIG. 3B) shows a prototyped PDMS chip. (FIG. 3C) shows a HepG2 cell line seeded and grown inside the chip. (FIG. 3D) shows a population of porous microspheres loaded with 6-coumarin flow study in microfluidic vascular chip (scale bar—100 nm).

FIGS. 4A-E depict a drug eluting population of porous beads (FIGS. 4A-D). Porous microspheres loaded with 6-coumarin (as in FIG. 3) in a drug elusion study in microfluidic vascular prototype chip (scale bar—100 nm). (FIG. 4E) provides an example of release kinetics of 6-coumarine as a model drug from the beads as sampled in the chip outlet.

FIGS. 5A-C Provide (FIG. 5A) confocal microscopy image of PLGA porous microspheres fabricated with microfluidic focused-flow chip design. (FIG. 5B). Rat In-vivo model, injection of micropsherse to liver via heptic artery. (FIG. 5C). Rat liver observed with white hypoix regions.

FIG. 6 provides a schematic illustration of a preparation method of porous microparticles using either “batch” or the microfluidic technique, according to some embodiments of the invention.

FIGS. 7A-D provide SEM images of core shell porous microspheres. (FIGS. 7A-B) show polystyrene beads encapsulated in a porous PLGA shell. (FIG. 7C) shows polystyrene beads embedded in a porous PLGA shell. (FIG. 7D) shows polystyrene beads revealed after 3 minutes in a 0.1M NaOH medium.

FIGS. 8A-B provide images of microspheres according to some embodiments of the invention: POS 010- (FIG. 8A) 50 mg/ml BSA, saturated PVA 5%, dropped in PVA 5%) microspheres and FIG. 8B) PEG PLGA 15% (RGPd 50155).

FIG. 9 shows high magnitude SEM image of PEG PLGA 15% particles.

FIG. 10 provides an illustration of dual embolization involving exemplary attachment of nanoparticles on the microparticles scaffold, in this specific application TPZ nanoparticles are shown with negative charge, to adhere on a doxorubicin loaded porous microsphere with the positive charge.

FIGS. 11A-B provide TEM images of NP's loaded with Tirapazamine (polymer is PLGA 75:25).

FIGS. 12A-B depict analysis of size distribution using DLS of the TPZ loaded NP's before and after lyophilization. FIG. 12A—before lyophilization at 168 nm. FIG. 12B—after lyophilization at 194 nm.

FIGS. 13A-C provides exemplary SEM images in high magnitude showing the surface of TPZ NP's after resuspension in water. The cryoprotectant used in freezing the sample was 20% trehalose.

FIGS. 14A-B provide SEM images showing the attached nanoparticles on the surface of the microparticles, the TPZ negatively charged NPs adhered to the doxorubicin loaded microparticle surface.

FIGS. 15A-D show examples of SEM images of porous microspheres+TPZ NP's composed of PLGA 75:25).

FIGS. 16A-B provide images of Microfluidic chip that was 3D printed on glass slide for generating Janus particles.

FIG. 17 provides an illustration of a Janus particle. Half dome is drug X and the second half is drug Y.

FIG. 18 shows Janus particles encapsulating two drug-like fluorophores (GFP, Cy3) as imaged by fluorescent image in two channels (for green or red—excitation/emission). The obtained particles exhibit dual signal and are shown in high and lower (bottom image) magnitude.

FIG. 19 shows porous particles encapsulating contrast agents for X-ray imaging. Particles were made of different polymers (PLA-120K and PLGA 75:25) with a contrast agent (Omnipaque) or without it as control. Images were taken by X-ray imager. The lower tube shown contained a non-diluted free omnipaque solution without polymer.

FIGS. 20A-B show X ray images of iron oxide loaded nanoparticles made of two types of polymers PLGA 75:25 or PLA.

FIGS. 21A-D show SEM images (on top) and bright field light microscopy images of porous microspheres covered by iron oxide np's.

FIG. 22 shows an EDX analysis for detection of Fe (iron) in the nanoparticle sample.

FIGS. 23A-B provide a comparison between TIBA as a contrast agent versus Omnipaque showing advantage to the TIBA in various nanoparticles composed of various polymers. The structure of TIBA is shown.

FIGS. 24A-C provide SEM images of microparticles with attached nanoparticles loaded with TIBA, EDX detected only small traces of I₂.

FIGS. 25A-B show US guided injection of N1-S1 cell line intrahepticly to rat liver to induce cancer. After 10-14 days the tumor is available. The image depicts the CT guided procedure of injection.

FIGS. 26A-B show a rat is under anesthesia in supine position and ultrasound assisted view of a needle during the cell injection.

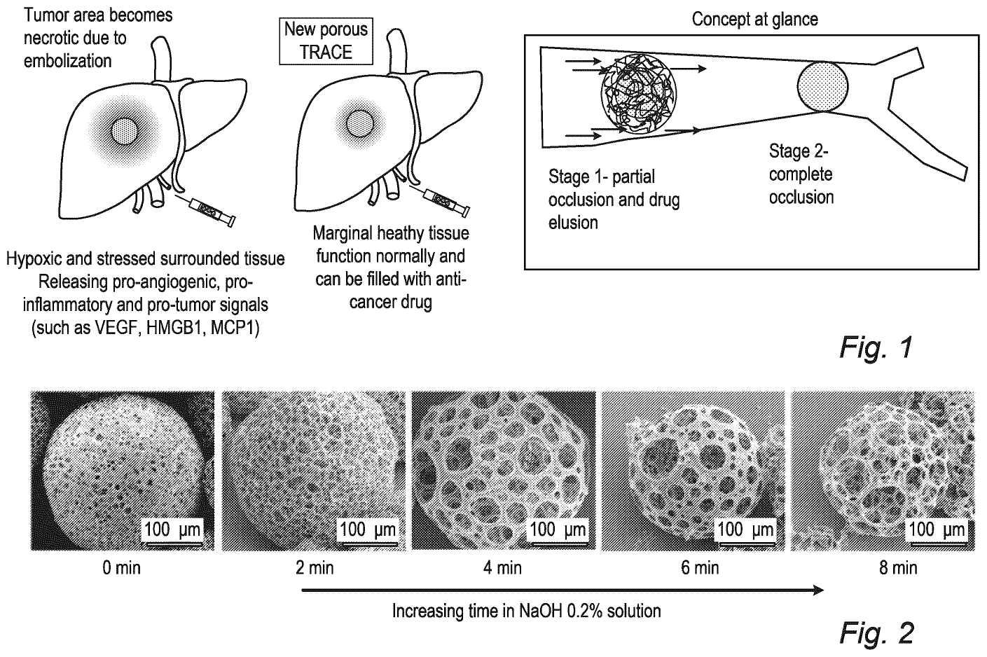

### DETAILED DESCRIPTION OF THE INVENTION

As provided herein, the invention generally provides a microparticle for use in a method of inducing or causing embolism to a blood microvessel in a subject, the microparticle having a plurality of flow through features and/or surface features permitting blood flow therethrough or on its surface and is of an average size selected to flow into microvessels in a subject body and occlude, clog or restrict blood flow to a target tissue.

The microparticle of the invention may be in a collapsible or erodible form capable of reduction in size to a size permitting partial or complete blood flow through the microvessels as compared to the microparticles' original or pre-collapsed form. The microparticle may have a first size (diameter) ranging between 10 and 500 μm and having a plurality of flow-through features selected from voids, spikes and channels, enabling blood flow through or around the features, wherein the microparticle is structured or configured to collapse or erode to microparticles of a second size being between 4 and 50 μm. The microparticle may be a core/shell structure having a solid core and a shell having a plurality of flow-through voids and/or surface features enabling flow of blood through the shell region. The microparticle may be surface decorated with a plurality of nanoparticles surface associated therewith. The microparticle may comprise one or more drugs or diagnostic agents. The microparticle may comprise or consist at least one polymeric material. The at least one polymeric material may be selected from poly (lactic-co-glycolic) acid (PLGA), poly (D,L-lactide) (PLA), polycaprolactone (PCL), poly(methyl methacrylate) (PMMA), poly(vinylacetate), polystyrene diblock copolymers, polymerized high internal phase emulsion (polyHIPE), polyvinyl alcohol (PVA), poly(N-isopropylacrylamide) (PNIPAAm), collagen, cellulose, alginate and gelatin.

The invention further provides an embolic device that is a microparticle according to the invention.

Also provided is a method of killing cancer cells or a tumor in a subject, the method comprising administering to said subject by parenteral administration a formulation comprising microparticles according to the invention.

A method for inducing embolism to a blood capillary or microvessel in a subject, is also provided, wherein the method comprising administering to said subject by parenteral administration a formulation comprising microparticles according to the invention.

Further provided is a method of transarterial chemoembolization (TACE), the method comprising administering to a blood vessel of a subject a therapeutically effective amount of the microparticles.

A method may be also be for selective eradication or killing or causing death to tumor cells in a subject, the method comprising causing hypoxia to said tumor cells or tissue containing same and administering to said subject (1) a population of microparticles, the population of microparticles comprising microparticles loaded with at least one hypoxia-activated agent; or (2) a population of microparticles, and subsequently thereto administering at least one hypoxia-activated agent; wherein the hypoxia activated agent is activated for eradicating the tumor cells at the region of hypoxia within a microvessel to the tumor. The hypoxia may be caused by administering an embolic device according to the invention. The method may comprise administering to said subject a population of microparticles comprising microparticles loaded with at least one hypoxia-activated agent; or administering to said subject a population of microparticles, and subsequently thereto administering at least one hypoxia-activated agent; wherein the hypoxia activated agent becomes activated for eradicating the tumor cells at the region of hypoxia within a microvessel to the tumor. In a method of the invention, the subject may be administered a population of microparticles, the population of microparticles comprising microparticles loaded with at least one hypoxia-activated agent. The method may be such that the subject is administered with a population of microparticles, and after a period of time, is administered with at least one hypoxia-activated agent. The at least one hypoxia-activated agent may be at least one hypoxic cytotoxin, e.g., tirapazamine (TPZ), banoxantrone (AQ4N), porfitomycin, apaziquone (EO9), 1,2-bis(methylsulfonyl)-1-(2-chloroethyl)-2-[[1-(4-nitrophenyl)ethoxy]carbonyl] hydrazine (KS 119), dinitrobenzamide mustard derivative and 4-[3-(2-nitro-1-imidazolyl)-propylamino]-7-chloroquinoline hydrochloride (NLCQ-1, NSC 709257). The at least one hypoxia-activated agent may be administered in combination with at least one anticancer drug.

The microparticle may be any of the microparticles discussed herein, e.g., may be selected from:
Microparticles having flow through features, wherein the microparticles are optionally loaded with or associated to one or more drug or diagnostic agent;
Microparticles having surface features, wherein the microparticles are optionally loaded with or associated to one or more drug or diagnostic agent;
Core/shell microparticles, each having a core and a shell, wherein the core is of a solid material, optionally biodegradable, and wherein the shell having flow through features or surface features permitting blood flow through or on the surface of the microparticles, wherein the microparticles are optionally loaded with or associated to at least one drug or diagnostic agent;
Microparticles surface decorated with one or more nanoparticles, wherein the microparticles and/or the nanoparticles are optionally loaded with or associated to at least one drug or diagnostic agent; and
Mixed microparticle populations comprising two or more of said microparticles.

The microparticles may be associated with at least one drug or at least one diagnostic agent. The drug may be selected amongst cytotoxic agents or cytostatic agents. The drug may be an anticancer drug, cytotoxic agent, a drug that selectively acts in hypoxic tumors, an anti-angiogenic agent, an anti VEGF agent, an antimetabolite, a topoisomerase inhibitor, a protein tyrosine kinase inhibitor, or a proteasome inhibitor. The drug may be selected amongst antimetabolites, topoisomerase inhibitors, vinca alkaloids, taxanes, platinum agents, antibiotics, alkylating agents, protein tyrosine kinase inhibitors, proteasome inhibitors, and antibodies. The drug may be selected from altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, carboplatin, cisplatin, eptaplatin, miriplatine hydrate, oxaliplatin, lobaplatin, nedaplatin, picoplatin, satraplatin, lobaplatin, nedaplatin, picoplatin, satraplatin, amrubicin, bisantrene, decitabine, mitoxantrone, procarbazine, trabectedin, clofarabine, amsacrine, brostallicin, pixantrone, laromustinel, etoposide, irinotecan, razoxane, sobuzoxane, teniposide, topotecan, amonafide, belotecan, elliptinium acetate, voreloxin, cabazitaxel, docetaxel, eribulin, ixabepilone, paclitaxel, vinblastine, vincristine, vinorelbine, vindesine, vinflunine; fosbretabulin, tesetaxel, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur, doxifluridine, elacytarabine, raltitrexed, sapacitabine, bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin, aclarubicin, peplomycin, pirarubicin, abarelix, abiraterone, bicalutamide, buserelin, calusterone, chlorotrianisene, degarelix, dexamethasone, estradiol, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol, acolbifene, danazol, deslorelin, epitiostanol, orteronel, aminoglutethimide, anastrozole, exemestane, fadrozole, letrozole, testolactone, formestane, crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib, talaporfin, temoporfin, alemtuzumab, besilesomab, brentuximab vedotin, cetuximab, denosumab, ipilimumab, ofatumumab, panitumumab, rituximab, tositumomab, trastuzumab, bevacizumab, pertuzumab, catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab, nivolumab, denileukin diftitox, ibritumomab tiuxetan, iobenguane, prednimustine, trastuzumab emtansine, estramustine, gemtuzumab, ozogamicin, aflibercept, edotreotide, inotuzumab ozogamicin, naptumomab estafenatox, and oportuzumab monatox. The drug may be doxorubicin.

The drug may be a hypoxic cytotoxin selected to act on tumors in hypoxic state (oxygen deprived state). The drug may be selected from tirapazamine (TPZ), banoxantrone (AQ4N), porfitomycin, apaziquone (EO9), 1,2-bis(methylsulfonyl)-1-(2-chloroethyl)-2-[[1-(4-nitrophenyl)ethoxy]carbonyl]hydrazine (KS 119), dinitrobenzamide mustard derivative (such as PR 104) and 4-[3-(2-nitro-1-imidazolyl)-propylamino]-7-chloroquinoline hydrochloride (NLCQ-1, NSC 709257). The drug may be two or more drugs, one being an anticancer drug and another a hypoxic cytotoxin. The anticancer drug may be doxorubicin and the hypoxic cytotoxin is TPZ.

The diagnostic material may be a contrasting agent or a radiopharmaceutical. The diagnostic material may be an X-ray contrasting agents, optionally selected from magnetite, iron-containing materials and Lipiodol; a magnetic resonance imaging agent optionally selected from gadoterate, gadobutrol, gadoteridol, gadopentetate, gadobenate, gadopentetic acid dimeglumine, gadoxentate, gadoversetamide, gadodiamide, albumin-binding gadolinium complexes, gadofosveset, gadocoletic acid, polymeric gadolinium complexes, gadomelitol, gadomer, gadoxetic acid; an ultrasound contrast agent optionally selected from microbubbles, perflutren lipid microspheres, octafluoropropane gas core with an albumin shell microbubbles, sulfur hexafluoride microbubbles, air and a lipid/galactose shell microbubbles, perflexane lipid microspheres. The diagnostic material is lipiodol.

The microparticles may be adapted for parenteral administration. The microparticles may be adapted for intramuscular (IM), subcutaneous (SC) or intravenous (IV) administrations. The microparticles may be administered IV.

A kit is provided which comprises a plurality of microparticles according to the invention and instructions of use. The kit may comprise microparticles that are provided as a powder or in an emulsion, dispersion or suspension. The microparticles may be selected from:
Microparticles having flow through features, wherein the microparticles are optionally loaded with or associated to one or more drug or diagnostic agent;
Microparticles having surface features, wherein the microparticles are optionally loaded with or associated to one or more drug or diagnostic agent;
Core/shell microparticles, each having a core and a shell, wherein the core is of a solid material, optionally biodegradable, and wherein the shell having flow through features or surface features permitting blood flow through or on the surface of the microparticles, wherein the microparticles are optionally loaded with or associated to at least one drug or diagnostic agent;
Microparticles surface decorated with one or more nanoparticles, wherein the microparticles and/or the nanoparticles are optionally loaded with or associated to at least one drug or diagnostic agent; and
Mixed microparticle populations comprising two or more of said microparticles.

A pharmaceutical or diagnostic formulation is provided which comprises an effective amount of microparticles of the invention. The formulation may be adapted for parenteral administration. The formulation may be adapted for intramuscular (IM), subcutaneous (SC) or intravenous (IV) administrations. The formulation may be an IV formulation, e.g., presented as an intravenous fluid.

A specific use of the flow-through drug loaded microspheres is for the occlusion of tumor liver blood vessels, while reducing the generation of acute hypoxic environment and tissue stress, resulting in lower level of pro-angiogenic factors releasing to tumor bed (such as VEGF, HIF-1 alpha) and hence improve clinical outcome and reduce cancer recurrence. By a non-limiting example, the particles are fabricated from poly lactic co-glycolic acid (PLGA), an FDA biocompatible and biodegradable material.

The flow-through microspheres structure causes a gradual flow reduction (compared to acute blockage in current “whole/solid/non-porous” beads) while enabling drug present in the microsphere and release therefrom to distally and selectively reach the tumor bed due to the porous mesh and the enlarged surface-volume ratio.

### Combinatory Slow-Release Hepatic Treatment

An embodiment of the invention is Janus microspheres, which can release two drugs simultaneously (FIG. 2). We use a focused flow or co-flowing or Y junction microfluidic chip design. Focused-Flow chip design consists of a cross junction, where the drug and polymer organic solution dispersed phase enter through a central channel and squeezed at the orifice by a continuous aqueous phase. The laminar flow (Re<100) through the orifice forces two different dispersed phases to form a parallel stream inside the central channel and a controlled droplets break-up by the vertical co-flowing of the continuous phase yielding monodisperse microemulsions. For oil (O)-in water (W) systems that are commonly used for drug encapsulation, the W continuous phase imposes shear force that tears discontinue phase O into a stream of micron-sized monodispersed O/W droplets. The droplets generally show much higher precision compared with those obtained by conventional methods. Janus spheres are particles that characterized with two or more distinct physical or chemical properties. We embed the drug inside the polymer during the fabrication process as described above) the fabricated porous microspheres with two drugs, one is cytotoxic (e.g. Doxorubicin).

PLGA microspheres loaded with DOX and TPZ were prepared using a microfluidic flow-focused chip design based on an adjusted solid-in-oil-in-water (S/O/W) method 120 mg of PLGA 75:25 polymer was dissolved in 2 mL of DCM and gently poured into a glass vessel containing DOX TPZ at various molar ratios dissolved in 1 mL dimethyl sulfoxide (DMSO). For solvents evaporation, the glass vial was held by tongs and its bottom was dipped inside a warm water (60° C.) bath, and a gentle nitrogen gas stream was applied from above 5 h. Next, the DOX-TPZ-polymer film was dissolved in 4 mL DCM and homogenized (MICCRA homogenizer disperser D-9, Heitersheim, Germany) with 1 mL of 1% (w/v) ABC solution for 3 min at 6000 rpm for fabrication of porous MS. The porosity was achieved by a gas-foaming technique using ammonium bicarbonate as a gas-foaming agent at the primary emulsion formation (O/W). Then, the homogenized solution was gently perfused into the microfluidic droplet generation chip using a glass syringe. Once the double emulsion formed, small micro gas-bubbles (carbon dioxide and ammonia gas bubbles) spontaneously appear during the solvent evaporation process. The flow-focused chip design consisted of a cross junction, in which the primary homogenized emulsion entered through a central channel and was squeezed at the orifice by two parallel streams of 1% (w/v) PVA solution to form a controlled droplet break-up. The fabricated MS were stirred in the chemical hood with an overhead propeller at 400 rpm overnight to ensure complete organic solvent evaporation. The MS were washed 3 times with DDW and centrifuged at 8000 rpm for 3 min. Finally, to prepare solidified particles, the MS pellet was resuspended with DDW and frozen overnight at −80° C. and lyophilized (Freezone 6 plus, Labconco, Kansas City, MO, USA) to produce a dry powder of MS for further storage (−20° C.) and characterization. Control microspheres (blank, without drugs) and microspheres loaded with DOX (MS-D) or TPZ (MS-P) were prepared in a similar way.

### Ex Vivo Model for Micro-Embolism

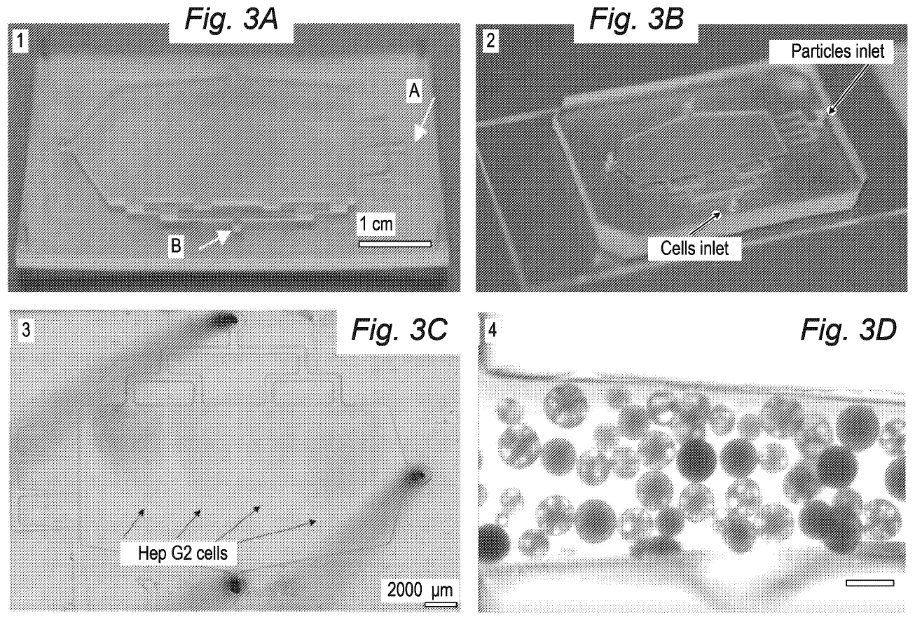

In order to be able to optimize the different formulations and to characterize their mechanical properties and release kinetics, an ex-vivo model for hepatic embolization based on microfluidic device (FIG. 3) was developed. For this, a microvascular construct was prototyped in polydimethylsiloxane (PDMS) and was connected to advanced flow system platform.

In the preliminary experiments, microspheres were introduced into the ‘vascular’ microfluidic device at an appropriate flow rate and time-lapse images were taken showing the formation of occlusions at the bifurcation within seconds of administration.

### In-Vivo Model

Alongside in-vitro and ex-vivo studies, an in-vivo model is also established. In this model, we induce liver cancer in rats and after adequate growth, we inject the particles being either the particles of the invention or solid non-porous particles and examine the effect of morphology (porous microspheres VS. non-porous) on liver tissue microenvironment stress proteins regulation (hypoxia-inducible factor-1α (HIF-1α), CRP-c reactive protein, heat shock protein 90 (HSP90) and changes in the level of vascular endothelial growth factor [VEGF] pro-angiogenic factor.

The particle's porosity degree might affect the tissue ischemia process due to potentially lower degree of blood occlusion, leading to a less “stressed” liver microenvironment compared to the non-porous microspheres. Moreover, the porous mesh structure will allow a distal diffusion of the drug to the tumor bed.

This strategy could be further expanded to treat other types of cancers, such as prostate, uterus and kidneys cancers.

### Porous Particle Synthesis

### Preparation of Water-in-Oil Emulsions

Porous MPs were prepared by the double emulsion batch method or via a microfluidic flow-focused chip design. Briefly, a given amount of polymer and 10 mg of 6-coumarin (green fluorescence, drug like molecule) were dissolved into a non-polar solvent (e.g., DCM, CF) or a polar solvent (e.g., EA). Then, two ml of 1% w/v ABC aqueous solution were added to the polymer solution. This mixture was homogenized with MICCRA homogenizer disperser D-9 (Heitersheim, Germany) at 11,000 rpm for 3 min to form the primary emulsion (W1/O). Then, the primary emulsion was introduced instantly to either a vessel of 0.5% (w/v) PVA solution or to a glass syringe to use the microfluidic droplet generation chip.

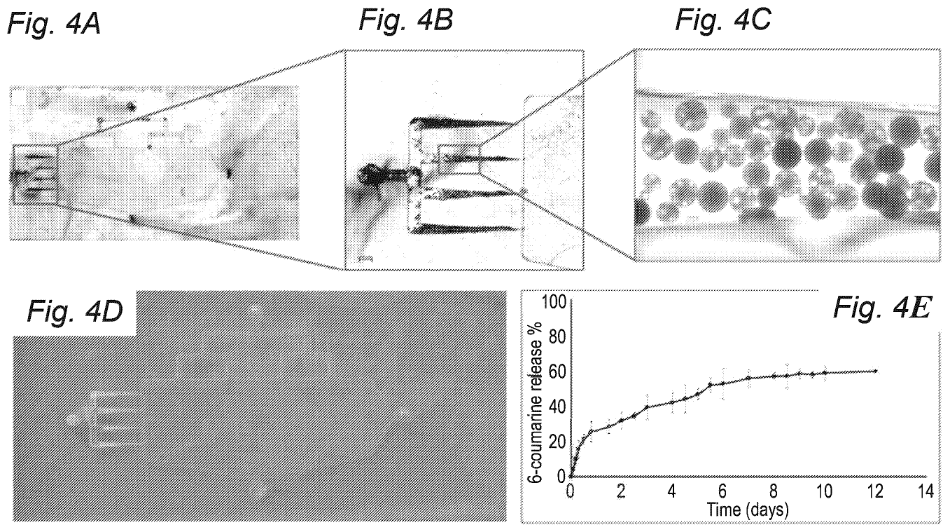

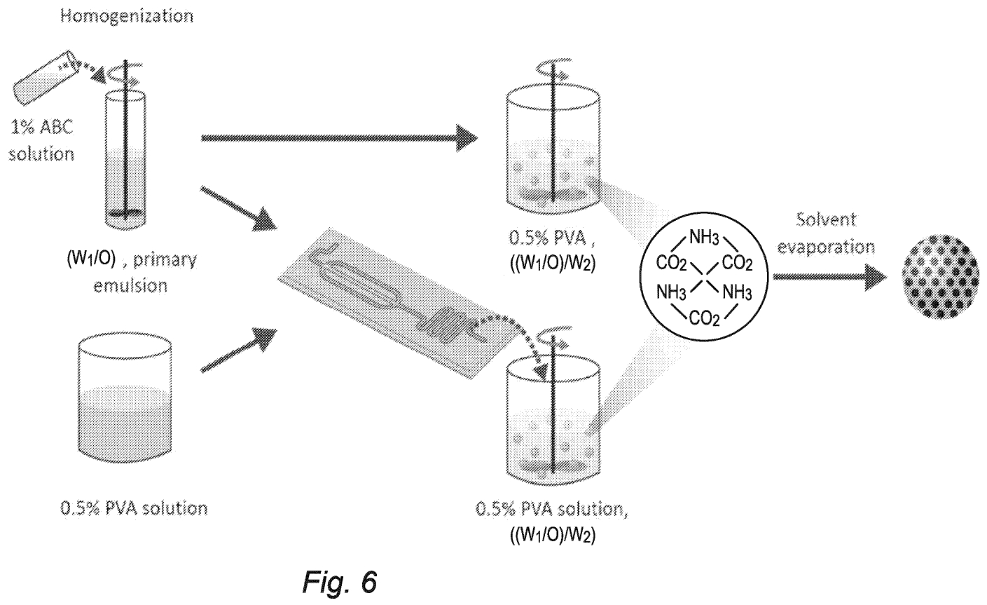

FIG. 3 shows the porous particles after their injection to flow device that mimic the in vivo conditions. The porous particles are “stuck” in the entrance channels and induce gradual (for porous) or acute (for non porous) hypoxia in the cells that resides in the central chamber of the chip. FIG. 4 shows the release kinetics of the loaded compounds on the embolic mimicking chip showing slow release of 6-coumarine loaded in microspheres. FIG. 6 shows a 6-coumarine loaded porous particles and their ischemic effect on the injected liver tissue in rat.

### Porous Microspheres Using Microfluidics

W/O emulsion was formed as detailed above and the primary (W1/O) emulsion was gently perfused into the microfluidic flow-focused chip using a glass syringe. The flow-focused chip design consisted of a cross junction, where the primary emulsion (W1/O) entered through a central channel and was squeezed at the orifice by a continuous aqueous phase of 0.5% (w/v) PVA solution to form a controlled droplet break-up of the secondary emulsion ((W1/O)/W2). The double emulsion was stirred with an overhead propeller at 600 rpm for 4 h to ensure complete evaporation of the organic solvent. The MPs were washed with DDW and centrifuged at 3000 rpm for 2 min to eliminate adsorbed PVA. Subsequently, the washed MPs were immersed in an aqueous NaOH (0.2 M) solution in predetermined time and washed thoroughly three times with DDW to remove any NaOH residues. Finally, to prepare solidified particles, the solution of washed particles was frozen overnight in −80° C. and lyophilized (Freezone 6 plus, Labconco, Kansas city, MO, USA) to produce a dry powder of particles that was stored at −20° C. The process is illustrated in FIG. 6.

### Porous Microspheres Using the Batch Method

The primary emulsion (W1/O) was instantly poured to 250 ml of 0.5% (w/v) aqueous PVA solution with an overhead propeller, stirring at 600 rpm for 4 h at the chemical hood to allow evaporation of the solvent from the secondary emulsion ((W1/O)/W2) to form hardened MPs. The steps previously described to produce the final MPs were followed. The process is illustrated in FIG. 6.

### Core-Shell Porous Particle Synthesis

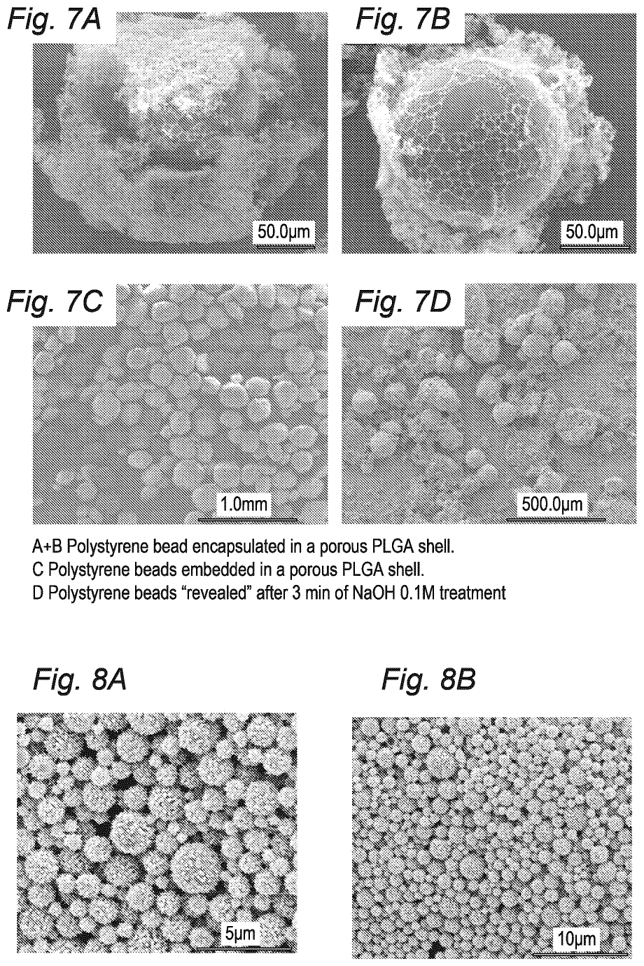

Porous MPs were prepared by the double emulsion method or via a microfluidic flow-focused chip design. Briefly, a given amount of polymer and 10 mg of 6-coumarin (green fluorescence, drug like molecule) were dissolved into a non-polar solvent (e.g., DCM, CF). Then, two ml of 3% w/v ABC aqueous solution and a given amount of polystyrene beads or solid polymers such as PLA etc. were added to the polymer solution. This mixture was homogenized with MICCRA homogenizer disperser D-9 (Heitersheim, Germany) at 11,000 rpm for 3 min to form the primary emulsion (W1/O). Then, the primary emulsion was introduced instantly to either a vessel of 0.5% (w/v) PVA solution (FIG. 7).

Microparticles with Spikes

To obtain microsphere between 1 and 3 nm, the standard procedure was modified. The following conditions were found to be optimal. 100 mg PEG-PLGA were dissolved in 1800 μl DCM (organic phase). BSA or drugs were dissolved in 200 μl DDW (aqueous phase) and added to the polymer solution. The two phases were mixed using homogenizer for one min at max speed (22000 rpm) on ice. The emulsion was then transferred to 4 ml saturated PVA 5% and mix again using homogenizer for 40 seconds at 40% max speed on ice. The double emulsion was then drop into 50 ml PVA 5% under stirring (800 rpm). After five minutes stirring 2.5 ml cold isopropanol were added and the solution was stirred for an additional hour. The microspheres were centrifuged at 5000 rpm for 10 min and pellet was re-suspended into 50 ml DDW to washes the microspheres. A total of three washes were made, each one using 50 ml DDW. Aliquot was removed for size and zeta determination. The microspheres solution was then frozen and lyophilized. The powder obtained was submitted to SEM for imaging.

An example of spiky particles composed of PEG-PLGA 5% (RGPd 5055)—includes PLGA 50:50 is shown in FIGS. 8A-B. POS 010- (50 mg/ml BSA, saturated PVA 5%, dropped in PVA 5%) microspheres: PEG PLGA 15% (RGPd 50155) are shown in FIG. 9.

### Tirapazamine NP's Adsorption on Porous Microspheres

The purpose of this experiment was to try and encapsulate Tirapazamine (TPZ, which is an experimental anticancer prodrug that is activated to a toxic radical only at very low levels of oxygen (hypoxia)) in nanoparticles formulation. Solid tumors are known to be hypoxic. The combination of TPZ+DOXO or other cytotoxic drug enhances the efficacy of the treatment—due to both, the ‘classic’ tumor hypoxic microenvironment and the hypoxia that is actively activated as a result of the embolization treatment itself.

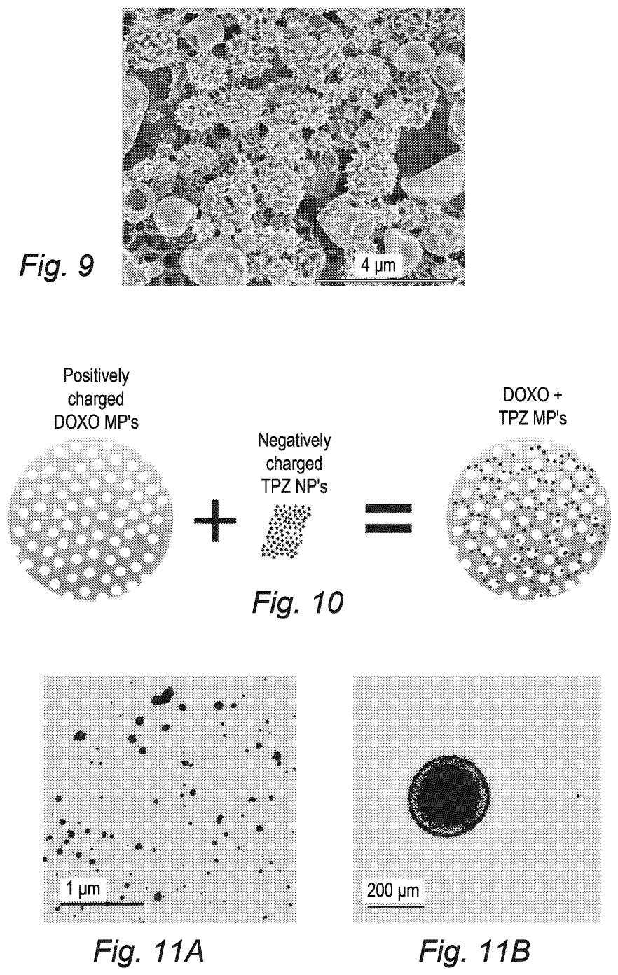

Therefore, the following systems were prepared:
A. Adsorption of TPZ NP's on the top of porous microspheres loaded with DOXO.
B. Fabrication of Janus microspheres (explanation below under ‘Janus exp’) (FIG. 10)

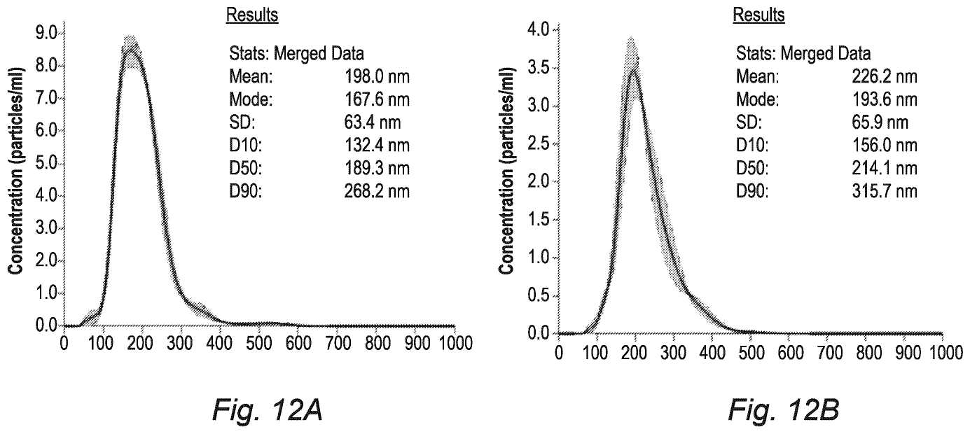

TEM images of NP's loaded with Tirapazamine (polymer is PLGA 75:25) are shown in FIG. 11. Analysis of TPZ loaded NP's before and after lyophilization is shown in FIGS. 12A-B.

SEM images of TPZ NP's after resuspension in DDW. The cryoprotectant we used in freezing the sample was 20% trehalose. After it freezes, we lyophilized it to get a white fine powder.

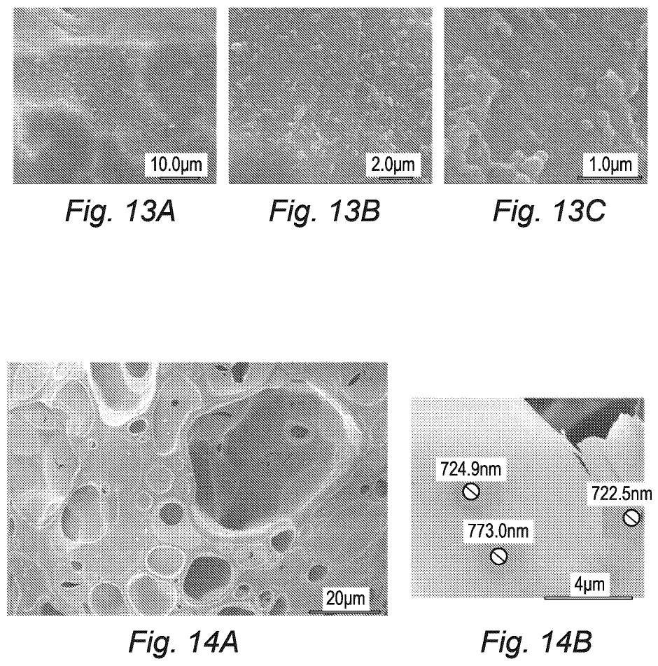

FIGS. 13A-C show a clear intact polymeric surface post lyophilization and attached nanoparticles that remained adhered to the surface post drying. FIGS. 14A-B show the TPZ nanoparticles adhere to the porous microparticles.

The produced NP's are negatively charge. To adsorb them to the porous microspheres surface we performed the following experiment: We took 1 mL of porous microspheres and submerged them in 2% polyethyleneimine solution (PEI, high molecular weight) for 5 minutes.

Afterwards we washed them 3 times with DDW (Centrifugation 3000 rpm). Next, we added 1 mL of NP's to the washed porous microspheres for 5 minutes.

### Repeat the Washing Step.

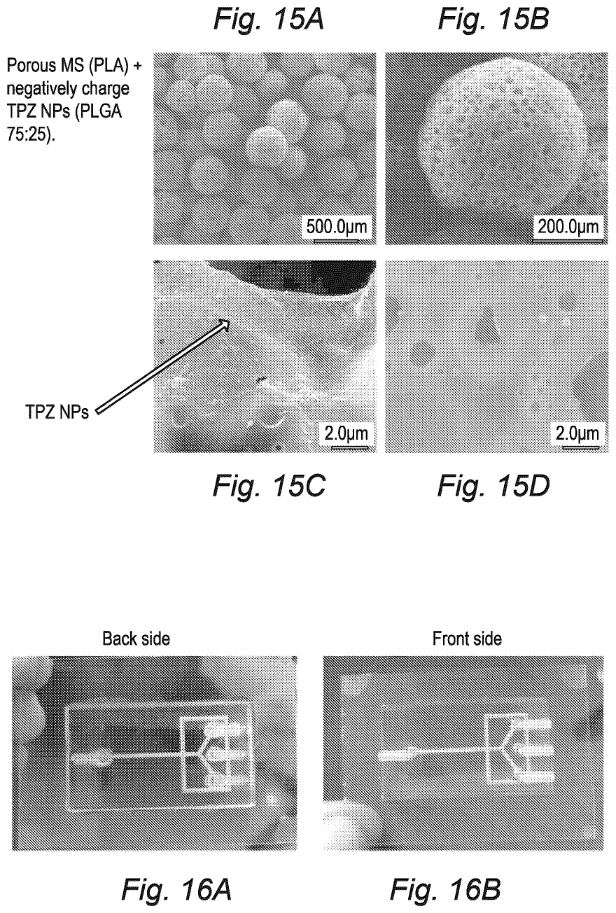

SEM images of porous microspheres with TPZ NP's formed on their outer layer are shown in FIGS. 15A-D.

### 3D Printed Janus Microfluidic Chip on Glass Slide

Janus droplets are spherical particles characterized with chemically and/or physically distinct parts/segments. This unique platform allows for example, to two different drugs to embed in the same particle.

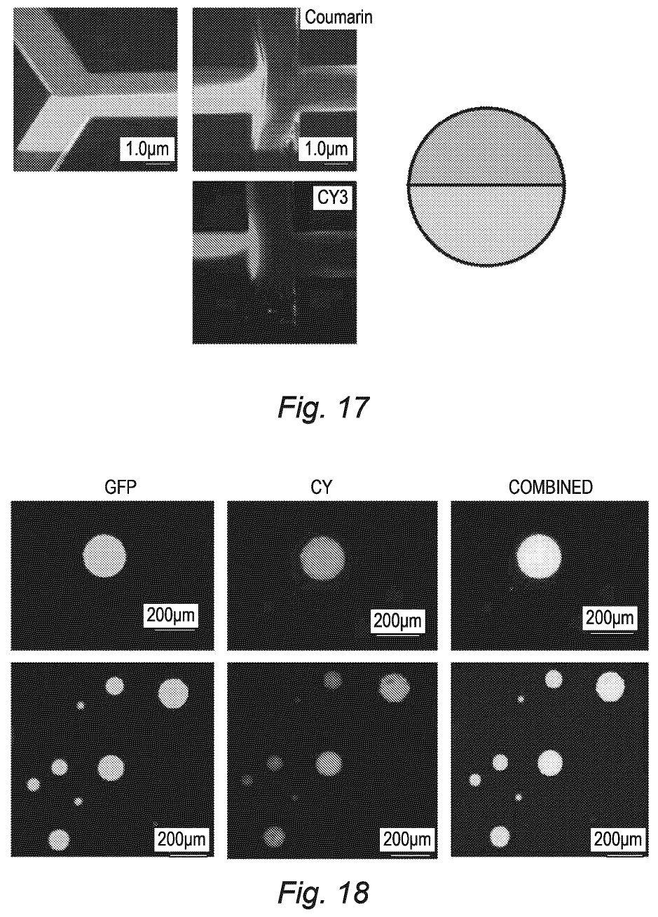

Microfluidic chip that was 3D printed on glass slide for Janus particles fabrication is shown in FIGS. 16A-B. The flow device enabled laminar co-flow of two types of polymers with various parameters and droplet generation enable the preparation of separated drops. Preliminary experiment: For Janus particles fabrication. The green is 6-coumarin (GFP). The orange color is DIL (CY3). It can be seen that there are two adjacent streams (orange is drug X, green is drug Y) that ‘cut’ by a vertical aqueous stream of 0.1% PVA solution. See FIGS. 17A-D and FIG. 18.

Fabricated Particles that can be Detected Under X-Ray Machine

The purpose of this experiment was to try to fabricate porous microspheres with the ability to be detected under fluoroscope machine (x-ray waves) thus, to be able to see the injected particles and the ‘embolized’ area during the procedure. Another benefit is to minimize the systemic toxicity by a local and precise administration.

Three different materials were used: Omnipaque, Iron-oxide and Triiodobenzoic acid (TIBA).

Preparation of Porous MS, Embedded with Contrast Agent (Iohexol, Omnipauge®).

Iohexol, sold under the trade name Omnipaque among others, is a contrast agent used during X-rays. This includes when visualizing arteries, veins, ventricles of the brain, the urinary system, and joints, as well as during computer tomography (Wikipedia).

In this experiment, omnipaque was used as the aqueous phase for the double emulsion preparation of the porous particles.

2% of ammonium bicarbonate were dissolved in omnipaque under vigorous vortex. Next the solution was homogenized with 3% PLGA 75:25/or PLA 120K solution. Then the first emulsion was added to PVA 0.1% solution to prepare the second emulsion and stirred it overnight under 400 rpm. A control ‘blank’ was also prepared without the omnipaque with PLGA 75:25.

In this slide we can see 4 different tubes. Particles prepared with omnipaque can be detected under fluoroscope scan. See FIG. 19.

Control are particles without Omnipaque. The second and third tubes contain porous particles that are made of different polymers (PLA-120K and PLGA 75:25) that contained omnipaque and an X-ray signal that was detected in fluoroscope machine. Fourth (bottom) tube is non-diluted omnipaque solution.

In this experiment, iron-oxide was used instead of omnipauqe. Same protocol as above.

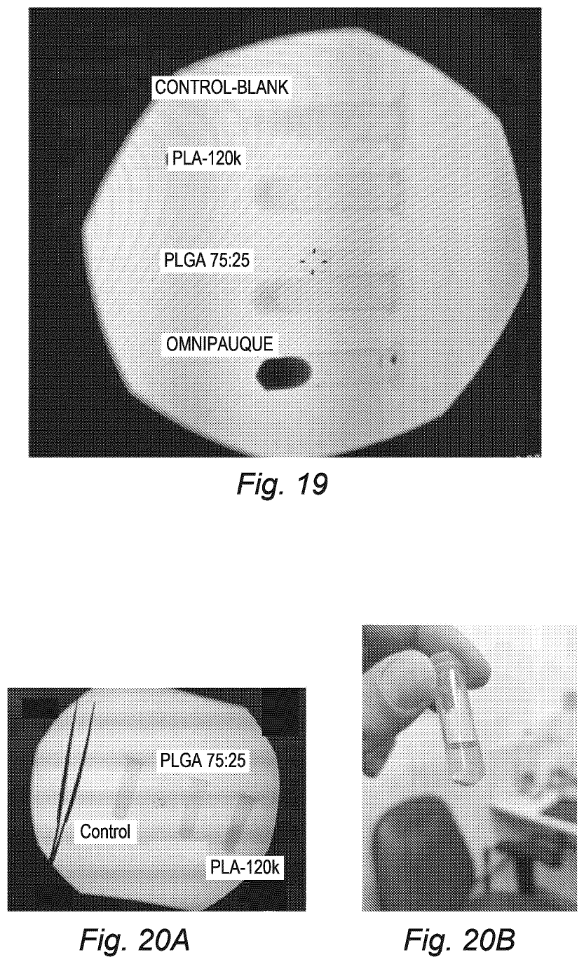

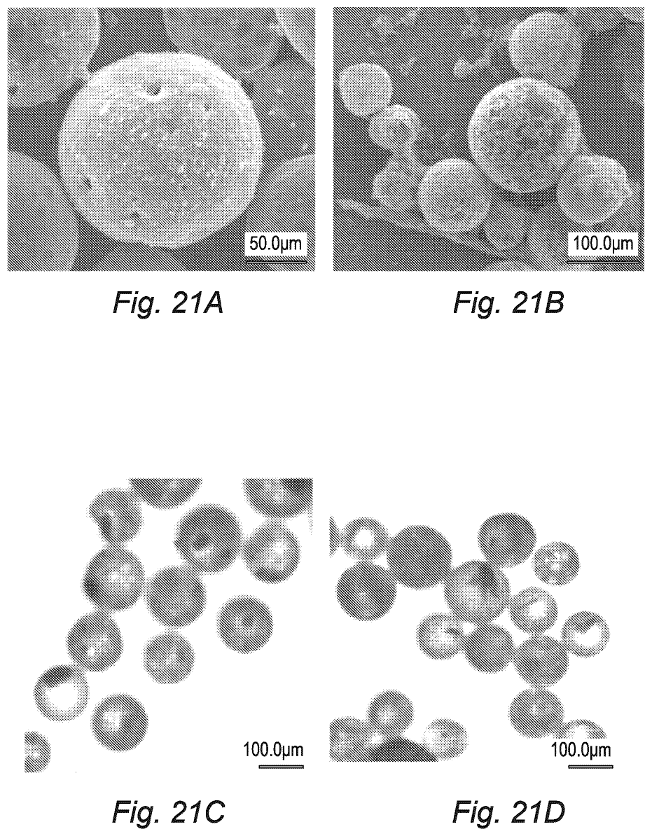

FIGS. 20A-B show the ability of iron oxide to be used for imaging under CT when they are loaded in polymer nanoparticles. FIGS. 21A-D show the microparticles decorated with iron oxide nanoparticles and their stability post lyophilization.

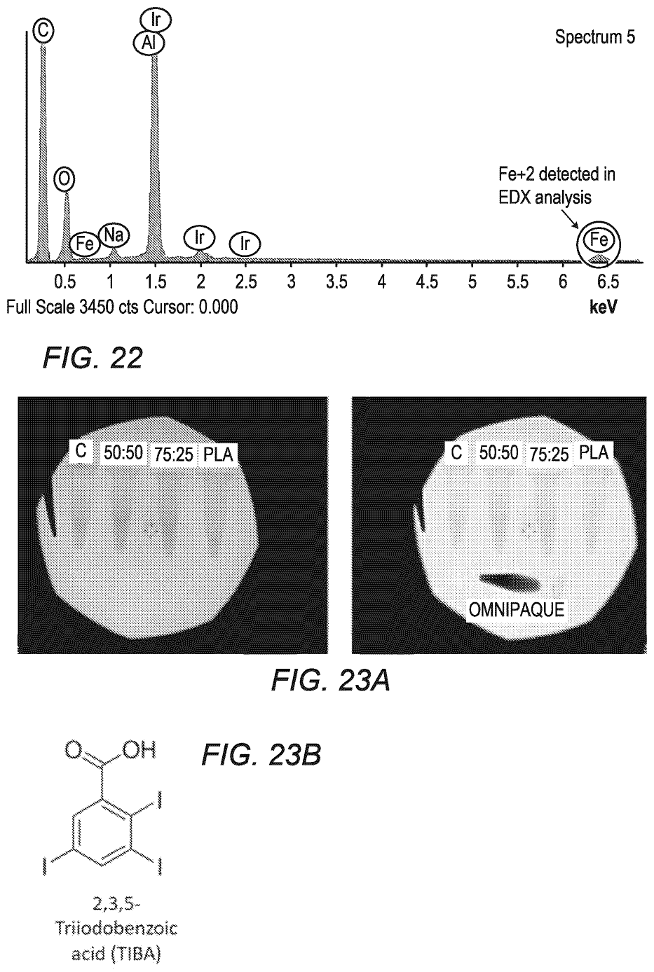

EDX test detected Fe (iron) in the sample. FIG. 22 shows successful loading of the iron oxide.

Another material that was tested (loaded in particles) was 2,3,5-Triiodobenzoic acid (TIBA). TIBA is contrast agent for computed tomography imaging (X-ray contrast agents). Fluoroscope images of TIBA particles are shown below. FIGS. 23A-B show images that depict the advantage of TIBA over omnipaque in generating high signal in CT.

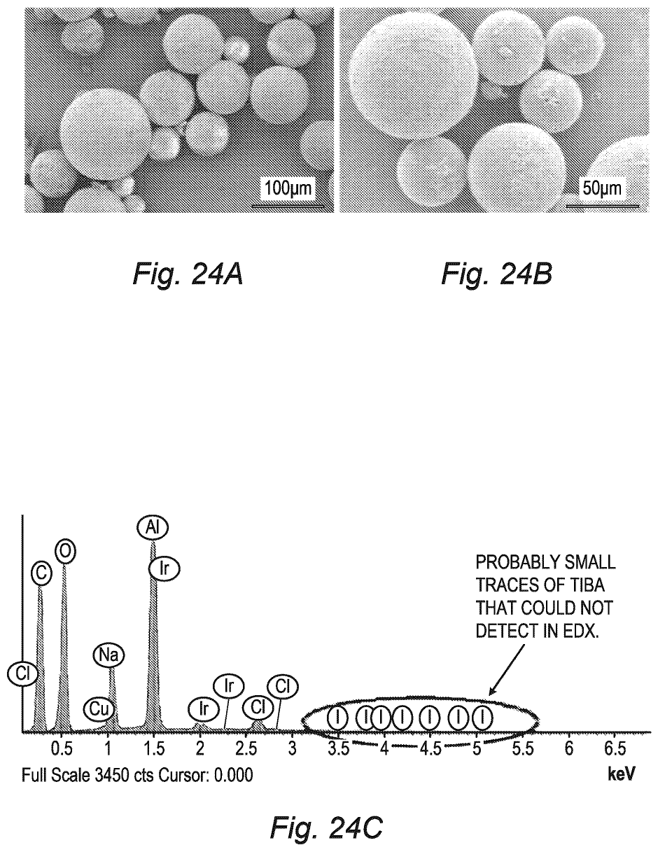

EDX detected only small traces of I₂. FIGS. 24A-C provide analytical confirmation of loading of TIBA.

### In-Vivo Model

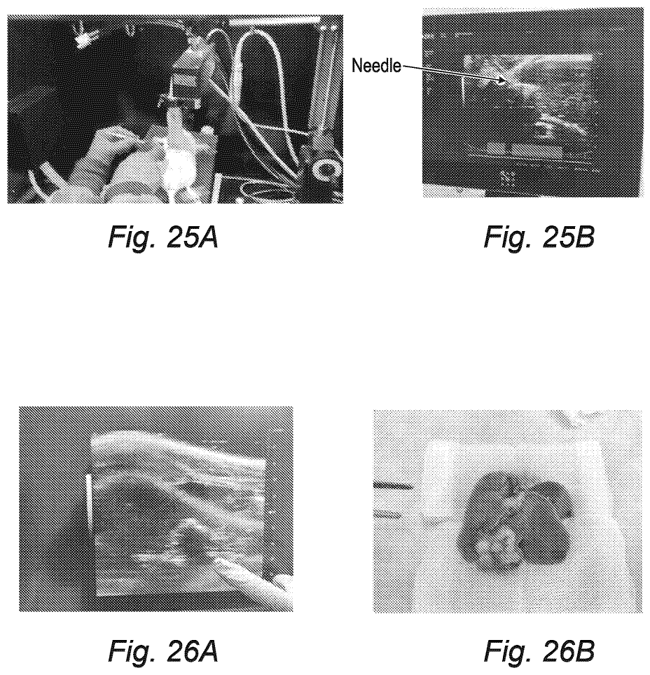

As depicted in FIGS. 25A-B, N1-S1 cell line (5×10\hat{ }6 cells in mixed solution of matrigel and PBS (50:50) were injected intrahepticly to a rat liver in order to induce cancer. After 10-14 days the tumor is available. Our next step is to embolize the tumor with our particles and hope to see improvement and tumor size reduction. As shown in FIGS. 26A-B, successful development of liver tumors was observed in the rat liver tissue 2 weeks post injection of N1-S1 cells.

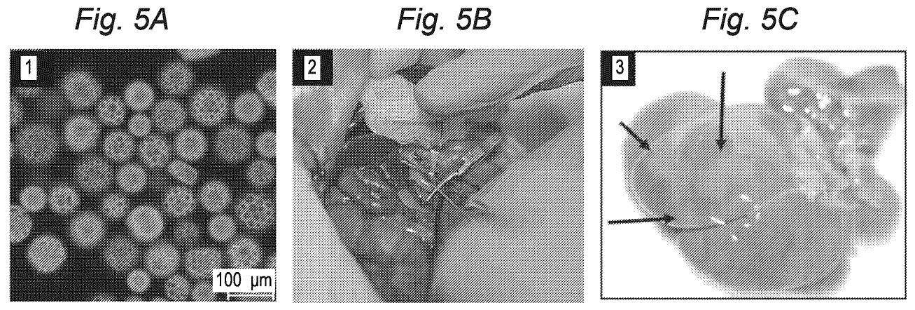

FIGS. 5A-C show confocal microscopy image of PLGA porous microspheres fabricated with microfluidic focused-flow chip design.

1-56. (canceled)

57. A microparticle for use in a method of inducing or causing embolism to a blood microvessel in a subject, the microparticle having a plurality of flow through features and/or surface features permitting blood flow therethrough or on its surface and is of an average size selected to flow into microvessels in a subject body and occlude, clog or restrict blood flow to a target tissue.

58. The microparticle according to claim 57 in a collapsible or erodible form capable of reduction in size to a size permitting partial or complete blood flow through the microvessels as compared to the microparticles' original or pre-collapsed form.

59. The microparticle according to claim 58, having a first size (diameter) ranging between 10 and 500 μm and having a plurality of flow-through features selected from voids, spikes and channels, enabling blood flow through or around the features, wherein the microparticle is structured or configured to collapse or erode to microparticles of a second size being between 4 and 50 μm.

60. The microparticle according to claim 57, being a core/shell structure having a solid core and a shell having a plurality of flow-through voids and/or surface features enabling flow of blood through the shell region.

61. The microparticle according to claim 57, being surface decorated with a plurality of nanoparticles surface associated therewith.

62. The microparticle according to claim 57 comprising one or more drug or diagnostic agent.

63. The microparticle according to claim 57, comprising or consisting of at least one polymeric material.

64. An embolic device being a microparticle according to claim 57.

65. A method for inducing embolism to a blood capillary or microvessel in a subject, the method comprising administering to said subject by parenteral administration a formulation comprising microparticles according to claim 57.

66. A method of transarterial chemoembolization (TACE), the method comprising administering to a blood vessel of a subject a therapeutically effective amount of microparticles according to claim 57.

67. A method of selective eradication or killing or causing death to tumor cells in a subject, the method comprising causing hypoxia to said tumor cells or tissue containing same and administering to said subject (1) a population of microparticles according to claim 57, the population of microparticles comprising microparticles loaded with at least one hypoxia-activated agent; or (2) a population of microparticles according to claim 57, and subsequently thereto administering at least one hypoxia-activated agent; wherein the hypoxia activated agent is activated for eradicating the tumor cells at the region of hypoxia within a microvessel to the tumor.

68. The method according to claim 67, wherein hypoxia is caused by administering embolic device in a form of a microparticle having a plurality of flow through features and/or surface features permitting blood flow therethrough or on its surface and is of an average size selected to flow into microvessels in a subject body and occlude, clog or restrict blood flow to a target tissue.

69. The method according to claim 67, wherein the at least one hypoxia-activated agent is at least one hypoxic cytotoxin.

70. The method according to claim 69, wherein the cytotoxin is selected from tirapazamine (TPZ), banoxantrone (AQ4N), porfitomycin, apaziquone (EO9), 1,2-bis(methylsulfonyl)-1-(2-chloroethyl)-2-[[1-(4-nitrophenyl)ethoxy]carbonyl]hydrazine (KS 119), dinitrobenzamide mustard derivative and 4-[3-(2-nitro-1-imidazolyl)-propylamino]-7-chloroquinoline hydrochloride (NLCQ-1, NSC 709257).

71. The method according to claim 70, wherein the at least one hypoxia-activated agent is administered in combination with at least one anticancer drug.

72. The method according to claim 67, wherein the microparticle is selected from:
microparticles having flow through features, wherein the microparticles are optionally loaded with or associated to one or more drug or diagnostic agent;
microparticles having surface features, wherein the microparticles are optionally loaded with or associated to one or more drug or diagnostic agent;
core/shell microparticles, each having a core and a shell, wherein the core is of a solid material, optionally biodegradable, and wherein the shell having flow through features or surface features permitting blood flow through or on the surface of the microparticles, wherein the microparticles are optionally loaded with or associated to at least one drug or diagnostic agent;
microparticles surface decorated with one or more nanoparticles, wherein the microparticles and/or the nanoparticles are optionally loaded with or associated to at least one drug or diagnostic agent; and
mixed microparticle populations comprising two or more of said microparticles.

73. The method according to claim 66, wherein the microparticles are associated with at least one drug or at least one diagnostic agent.

74. A pharmaceutical or diagnostic formulation comprising an effective amount of microparticles according to claim 57.

75. The formulation according to claim 74, adapted for parenteral administration.

76. The formulation according to claim 74, adapted for intramuscular (IM), subcutaneous (SC) or intravenous (IV) administrations.
